(19) United States
(12) Patent Application Publication
(10) Pub. No.: US 2026/0348771 A1
(43) Pub. Date: Nov. 5, 2026

(54) TEMPERATURE MEASUREMENT METHOD, TEMPERATURE MEASUREMENT DEVICE, MEDICAL DEVICE SYSTEM, AND LASER TREATMENT METHOD

(71) Applicant: OLYMPUS CORPORATION, Tokyo (JP)

(72) Inventors: Mitsuru NAMIKI, Tokyo (JP); Ikutoshi FUKUSHIMA, Tokyo (JP)

(73) Assignee: OLYMPUS CORPORATION, Tokyo (JP)

(21) Appl. No.: 19/737,634

(22) Filed: Jul. 10, 2026

Related U.S. Application Data
(63) Continuation of application No. 18/234,662, filed on Aug. 16, 2023, now Pat. No. 12,704,416.
Application No. 18/234,662 is a continuation of application No. PCT/JP2021/009590, filed on Mar. 10, 2021.

(51) Int. Cl. G01K 11/32; A61B 1/018; A61B 18/00; A61B 18/26

(52) U.S. Cl. CPC G01K 11/32; A61B 18/26; A61B 1/018; A61B 2018/00678; A61B 2018/00696; A61B 2018/00791

(57) ABSTRACT

Provided is a temperature measurement method for measuring an ambient temperature of a long-length medical device, using first and second optical fibers attached to the medical device, the first and second optical fibers extending in parallel with each other along a longitudinal direction of the medical device, and the first optical fiber having a distal end serving as a measurement unit extending beyond a distal end of the second optical fiber along a longitudinal direction of the first and second optical fibers. The method includes: measuring an optical path length difference between a first optical path including the first optical fiber and a second optical path including the second optical fiber; and calculating a temperature of the measurement unit based on the optical path length difference. When temperatures of the first and second optical fibers are uniform, optical path lengths of the first and second optical paths are equal.

### CROSS-REFERENCE TO RELATED APPLICATIONS

This is a continuation of U.S. patent application Ser. No. 18/234,662, filed on Aug. 16, 2023, which is a continuation of International Application PCT/JP2021/009590, filed on Mar. 10, 2021. The disclosures of U.S. patent application Ser. No. 18/234,662 and International Application PCT/JP2021/009590 are hereby incorporated by reference herein in its entirety.

### TECHNICAL FIELD

The present invention relates to a temperature measurement method, a temperature measurement device, a medical device system, and a laser treatment method.

### BACKGROUND ART

Conventional medical devices equipped with temperature sensors are known (see PTL 1, for example). There is a wide variety of temperature sensors such as optical temperature sensors, electrical temperature sensors, infrared temperature sensors, and fiber optic temperature sensors. Optical temperature sensors are best suited for medical devices in terms of measurement accuracy, fineness, and biocompatibility of the material. For example, electrical temperature sensors require a large space and their measurement values are easily fluctuated by external noise such as electromagnetic waves and high frequencies. Infrared temperature sensors have inadequate measurement accuracy, and infrared rays may be interfered with by therapeutic flashes emitted from medical devices, bubbles generated during treatment, etc.

The medical device in PTL 1 is equipped with a fiber Bragg grating (FBG) sensor, one of the optical temperature sensors. The FBG sensor is installed inside an optical fiber that extends along the outer surface of the medical device and measures the ambient temperature of the medical device.

### CITATION LIST

### Patent Literature

{PTL 1}Japanese Translation of PCT International Application, Publication No. 2016-517777

### Summary of Invention

A first aspect of the present invention is a temperature measurement method for measuring an ambient temperature of a long-length medical device, using a first optical fiber and a second optical fiber attached to the medical device, the first optical fiber and the second optical fiber extending in parallel with each other along a longitudinal direction of the medical device, and the first optical fiber having a distal end serving as a measurement unit extending beyond a distal end of the second optical fiber along a longitudinal direction of the first and second optical fibers, the method including: measuring an optical path length difference between a first optical path including the first optical fiber and a second optical path including the second optical fiber; and calculating a temperature of the measurement unit based on the optical path length difference, wherein when temperatures of the first optical fiber and the second optical fiber are uniform, an optical path length of the first optical path and an optical path length of the second optical path are equal.

A second aspect of the present invention is a temperature measurement device that measures an ambient temperature of a long-length medical device, including: a first optical fiber and a second optical fiber that are positioned in the medical device and extend in parallel with each other, the first optical fiber having a distal end serving as a measurement unit extending beyond a distal end of the second optical fiber along a longitudinal direction of the first and second optical fibers; an optical path length difference measuring unit that measures an optical path length difference between a first optical path including the first optical fiber and a second optical path including the second optical fiber; and a processor including hardware configured to calculate a temperature of the measurement unit based on the optical path length difference, wherein when temperatures of the first optical fiber and the second optical fiber are uniform, an optical path length of the first optical path and an optical path length of the second optical path are equal.

A third aspect of the present invention is a laser treatment method including: preparing a medical device equipped with a first optical fiber and a second optical fiber, the first optical fiber and the second optical fiber extending in parallel with each other and the first optical fiber having a distal end serving as a measurement unit extending beyond a distal end of the second optical fiber along a longitudinal direction of the first and second optical fibers; positioning the medical device with respect to a treatment target; projecting a laser beam from the medical device to the treatment target; and measuring an ambient temperature of the medical device, wherein the measuring includes: measuring an optical path length difference between a first optical path including the first optical fiber and a second optical path including the second optical fiber; and calculating a temperature of the measurement unit based on the optical path length difference, and when temperatures of the first optical fiber and the second optical fiber are uniform, an optical path length of the first optical path and an optical path length of the second optical path are equal.

### BRIEF DESCRIPTION OF DRAWINGS

FIG. 1 is an overall configuration diagram of a medical device system according to an embodiment of the present invention.

FIG. 2 is an overall configuration diagram of a temperature measurement device according to a first embodiment of the present invention.

FIG. 3A is a diagram showing the relationship between the distance between a split-and-combine unit and a coupler and the interference intensity before temperature change.

FIG. 3B is a diagram showing the relationship between the distance between the split-and-combine unit and the coupler and the interference intensity after temperature change.

FIG. 4 is a graph showing an example of the relationship between the amount of change in optical path length and the temperature of a measurement unit.

FIG. 5 is a diagram for explaining the principle of temperature measurement in the temperature measurement device.

FIG. 6 is a flowchart showing a laser treatment method.

FIG. 7 is an overall configuration diagram of a temperature measurement device according to a second embodiment of the present invention.

FIG. 8 is an overall configuration diagram of a temperature measurement device according to a third embodiment of the present invention.

FIG. 9A is a graph showing the relationship between the wavelength and the interference intensity before temperature change.

FIG. 9B is a graph showing the relationship between the wavelength and the interference intensity after temperature change.

FIG. 10 is an overall configuration diagram of a temperature measurement device according to a fourth embodiment of the present invention.

FIG. 11A is a diagram showing the relationship between the distance between an emitting end of a third optical fiber and a coupler and the interference intensity before temperature change.

FIG. 11B is a diagram showing the relationship between the distance between the emitting end of the third optical fiber and the coupler and the interference intensity after temperature change.

FIG. 12A is a partial configuration diagram of a modification of the medical device.

FIG. 12B is a partial configuration diagram of another modification of the medical device.

FIG. 12C is a partial configuration diagram of still another modification of the medical device.

### DESCRIPTION OF EMBODIMENTS

### First Embodiment

A temperature measurement device and medical device system according to the first embodiment of the present invention will now be described with reference to the drawings.

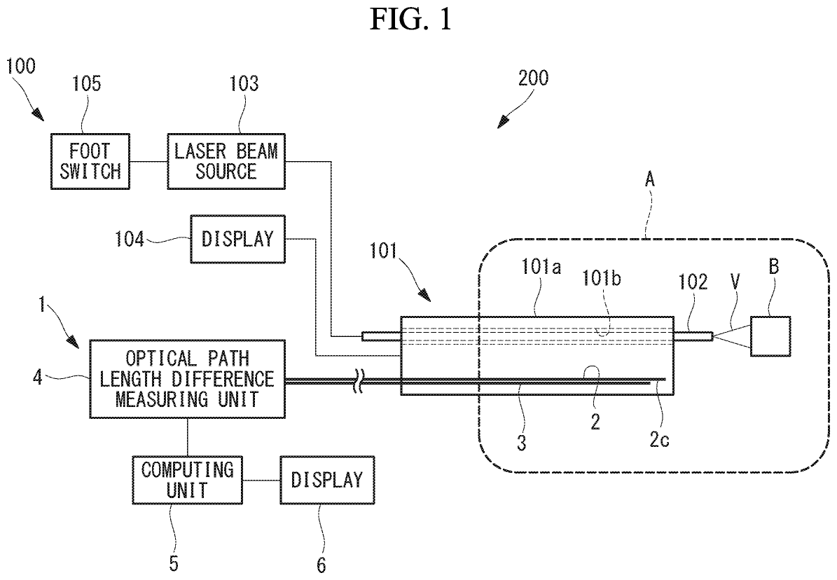

As shown in FIG. 1, the medical device system 200 is a laser treatment system that treats a treatment target B in a body A with a therapeutic laser beam V. The medical device system 200 includes a medical device 100, which is a laser treatment device.

The medical device 100 includes an endoscope 101, an optical fiber 102, a laser beam source 103, and a display 104. The endoscope 101 is, for example, a soft or rigid ureteroscope. The optical fiber 102 passes through a channel 101b in the long insertion portion 101a of the endoscope 101, the distal end of the optical fiber 102 protrudes from the distal end of the insertion portion 101a, and the proximal end of the optical fiber 102 is connected to the laser beam source 103. The medical device 100 may further include other components such as an image processor and a laser beam control unit, if necessary. The image processor processes endoscopic images, and the laser beam control unit controls the intensity, pulse waveform, frequency, and the like of the laser beam V which is supplied from the laser beam source 103 to the optical fiber 102. The display 104 is an optional display device such as a liquid crystal display. A perfusion device (not shown in the drawing) is provided to supply perfusion fluid to the body A via the channel 101b.

After the perfusion device fills the inside of the body A with perfusion fluid, the surgeon emits a laser beam V from the laser beam source 103 by, for example, stepping on a foot switch 105 connected to the laser beam source 103 while observing the endoscope image displayed on the display 104. The laser beam V enters the proximal end of the optical fiber 102 from the laser beam source 103 and is projected from the distal end of the optical fiber 102 onto the treatment target B. In one example, the laser beam V is an infrared laser beam that shatters the treatment target B, a stone. The infrared laser beam V achieves lithotripsy by thermal energy absorbed by water or perfusion fluid in the stone or voids of the stone, and the energy of the emitted laser beam V is stored as thermal energy in the entire body A. In other words, the irradiation with the laser beam V increases the temperature of the entire body A, including the perfusion fluid surrounding the distal end of the insertion portion 101a, the endoscope 101, and the optical fiber 102.

The medical device system 200 further includes a temperature measurement device 1 for measuring the temperature around the distal end of the insertion portion 101a. The temperature measurement device 1 includes a first optical fiber 2, a second optical fiber 3, an optical path length difference measuring unit 4 that measures the optical path length difference ΔL generated in the two optical paths via the optical fibers 2 and 3, respectively, a computing unit 5 that calculates the temperature based on the optical path length difference ΔL, and a display 6 that displays the temperature. The display 6 is an optional display device, such as a liquid crystal display, and may be common to the display 104.

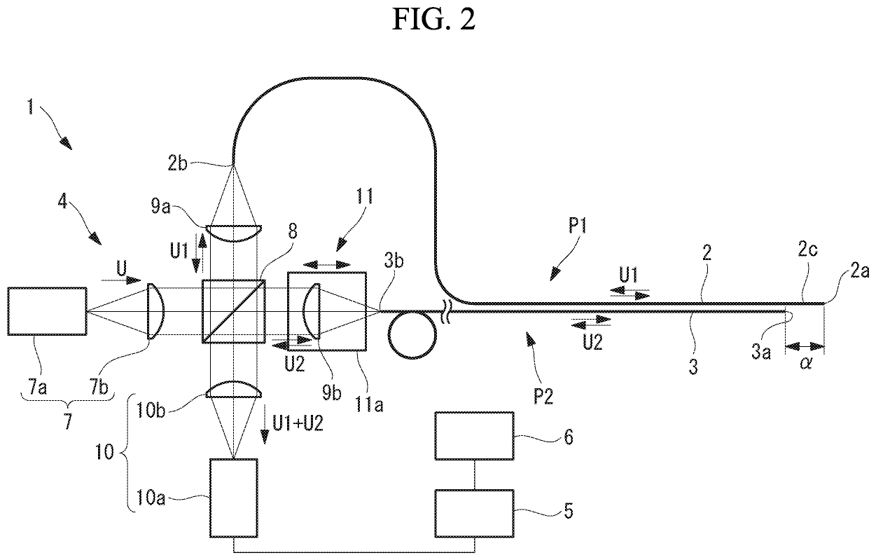

FIG. 2 shows the specific configuration of the temperature measurement device 1.

Each of the optical fibers 2 and 3 is a single-mode fiber having a cladding diameter of 125 μm, for example. The lengths of the two optical fibers 2 and 3 are equal or approximately equal. The two optical fibers 2 and 3 are close to each other in the radial direction of the optical fibers 2 and 3, extend in parallel along the longitudinal direction of the insertion portion 101a, and are attached to the side surface of the insertion portion 101a. The distal ends 2a and 3a of the two optical fibers 2 and 3 are located in the vicinity of the distal end of the insertion portion 101a, and the distal end 2a of the first optical fiber 2 is longer by a length a along the longitudinal direction of the optical fibers 2 and 3 than the distal end 3a of the second optical fiber 3. The distal end of the length a of the first optical fiber 2 is a measurement unit 2c, and the computing unit 5 calculates the temperature change amount T and temperature of the measurement unit 2c as described below.

The optical path length difference measuring unit 4 measures the optical path length difference ΔL between a first optical path P1 including the first optical fiber 2 and a second optical path P2 including the second optical fiber 3. To be specific, the optical path length difference measuring unit 4 includes a light source unit 7, a split-and-combine unit 8, two couplers 9a and 9b, a detection unit 10, and an optical path length adjustment mechanism 11.

The light source unit 7 has a low-coherence light source 7a that emits low-coherence light, which is measurement light U, and a collimating lens 7b that forms the measurement light U into collimated light.

The split-and-combine unit 8 is a spatial optical system, such as a beam splitter, and is positioned between the light source unit 7 and the proximal ends 2b and 3b of the two optical fibers 2 and 3. The split-and-combine unit 8 splits the measurement light U incident from the light source unit 7 into measurement light U1 traveling along the first optical path P1 and second measurement light traveling along the second optical path P2. The split-and-combine unit 8 also combines the first measurement light U1 that has reciprocated along the first optical path P1 and the second measurement light U2 that has reciprocated along the second optical path P2.

The first optical path P1 is the optical path between the split-and-combine unit 8 and the distal end 2a of the first optical fiber 2. In other words, the first measurement light U1 returns to the split-and-combine unit 8 from the split-and-combine unit 8 via the coupler 9a, proximal end 2b, distal end 2a, proximal end 2b, and coupler 9a in this order.

The second optical path P2 is the optical path between the split-and-combine unit 8 and the distal end 3a of the second optical fiber 3. In other words, the second measurement light U2 returns to the split-and-combine unit 8 from the split-and-combine unit 8 via the coupler 9b, proximal end 3b, distal end 3a, proximal end 3b, and coupler 9b in this order.

A reflective surface may be provided at each of the distal ends 2a and 3a to increase the reflectivity of the measurement light U1 and U2 at the distal ends 2a and 3a.

The couplers 9a and 9b are disposed between the split-and-combine unit 8 and the proximal ends 2b and 3b of the optical fibers 2 and 3, respectively, and optically couple the split-and-combine unit 8 and each of the proximal ends 2b and 3b. To be specific, the couplers 9a and 9b are convex lenses. The coupler 9a focuses the first measurement light U1, which is collimated light incident from the split-and-combine unit 8, to the proximal end 2b, and the coupler 9b focuses the second measurement light U2, which is collimated light incident from the split-and-combine unit 8, to the proximal end 3b.

The detection unit 10 includes a photodetector 10a that detects the interference intensity of the combined light U1+U2 of the measurement light U1 and the measurement light U2 combined by the split-and-combine unit 8. The detection unit 10 may further include a lens 10b disposed between the split-and-combine unit 8 and the photodetector 10a to focus the combined light U1+U2 onto the photodetector 10a.

The optical fibers 2 and 3 have mutually identical optical characteristics, the couplers 9a and 9b have mutually identical optical characteristics, and the optical path length L1 of the first optical path P1 and the optical path length L2 of the second optical path P2 are designed to be equal. In practice, however, it is not easy to make the two optical path lengths L1 and L2 equal with wavelength-order accuracy due to errors in the lengths of the optical fibers 2 and 3 and the like.

The optical path length adjustment mechanism 11 fine-tunes at least one of the optical path length L1 and the optical path length L2 with wavelength-order precision. The optical path length adjustment mechanism 11 has a fine movement stage 11a that can be moved along the longitudinal direction of the second optical fiber 3 with a resolution of a wavelength order. The coupler 9b and the proximal end of the second optical fiber 3 are fixed to the fine movement stage 11a, and the fine movement stage 11a, the coupler 9b, and the proximal end of the second optical fiber 3 move together. Consequently, the fine movement of the fine movement stage 11a can slightly change the distance d between the split-and-combine unit 8 and the coupler 9b, thereby fine-tuning the optical path length L2. The fine movement stage 11a can be either manual or motorized. In the case where it is motorized, the optical path length adjustment mechanism 11 further includes a motor (not shown in the drawings) that drives the fine movement stage 11a.

The aforementioned configuration of the optical path length adjustment mechanism 11 is merely one example, and the optical path length adjustment mechanism 11 may adjust the optical path length by other means. To give other examples, the optical path length adjustment mechanism 11 may be configured to adjust the optical path length by changing the thickness of a medium having one or more refractive indices. For example, the optical path length adjustment mechanism 11 may have two prisms whose sides are right-angled triangles and whose slopes face each other, and the thickness (optical path length difference) may be made variable by increasing or decreasing the overlap of the slopes of the two prisms.

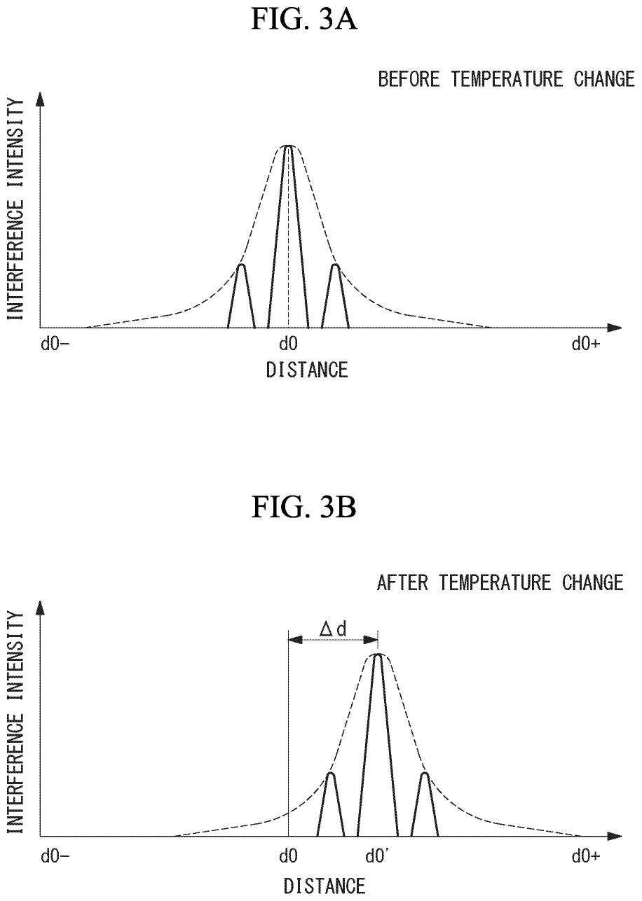

The wavelength of the first measurement light U1 and the wavelength of the second measurement light U2 are equal. In addition, the phase of the first measurement light U1 and the phase of the second measurement light U2 are identical at the split-and-combine unit 8, which is the starting point of the optical paths P1 and P2. For this reason, when the optical path lengths L1 and L2 are equal, as shown in FIG. 3A, the interference intensity distribution is generated by the mutual interference and mutual intensification of the two types of combined measurement light U1 and U2, and the interference intensity of the combined light U1+U2 detected by the photodetector 10a becomes high. Therefore, the optical path length L2 can be adjusted to the same length as the optical path length L1 by determining the distance d to be the reference distance d0 at which the interference intensity peaks.

The optical path length adjustment mechanism 11 may be configured to adjust the optical path length L1 instead of the optical path length L2, or to adjust both the optical path length L1 and the optical path length L2.

Here, when a temperature gradient is generated in optical fibers 2 and 3 due to a temperature change around the insertion portion 101a, the first optical path length L1 and the second optical path length L2 change. In this case, as explained later, an optical path length difference ΔL=|L1−L2| is generated according to the temperature change amount T of the measurement unit 2c, and the distance d at which interference occurs changes from the reference distance d0 by the amount of the optical path length difference ΔL. The reference distance d0 is the distance d at which the interference intensity peaks when the temperatures of the optical fibers 2 and 3 are uniform before the temperature change occurs in the insertion portion 101a. To be specific, as shown in FIG. 3B, when the temperature of the measurement unit 2c rises, the distance d at which interference occurs becomes larger than the reference distance d0, and when the temperature of the measurement unit 2c falls, the distance d at which interference occurs becomes smaller than the reference distance d0. Accordingly, the amount of temperature change T of the measurement unit 2c can be estimated from the amount of change Δd of the distance d at which interference occurs, from the reference distance d0. In FIGS. 3A and 3B, the horizontal axis represents the distance d between the split-and-combine unit 8 and the coupler 9b, and the vertical axis represents the intensity of the combined light U1+U2 detected by the photodetector 10a.

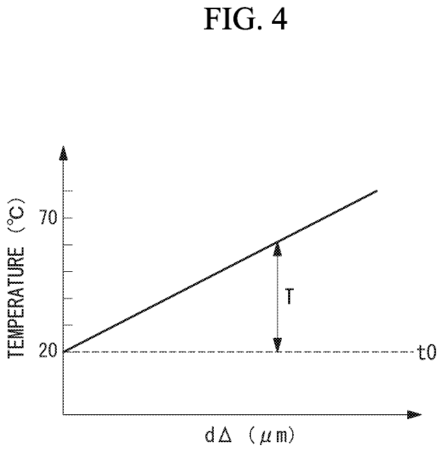

As shown in FIG. 4, the computing unit 5 stores an equation or table showing the relationship between the amount of change Δd and the amount of temperature change T. The equation or table is experimentally obtained. The computing unit 5 obtains the amount of change Δd and the corresponding amount of temperature change T from the equation or table. The vertical axis in FIG. 4 represents the temperature t of the measurement unit 2c calculated using the amount of temperature change T. The computing unit 5 may calculate the current temperature t of the measurement unit 2c by adding the amount of temperature change T to the temperature t0 of the measurement unit 2c which is obtained before the temperature change. The temperature t0 before the temperature change is, for example, the measured temperature of the optical fibers 2 and 3 before the insertion portion 101a is inserted into the body A, or the measured temperature of the temperature invariant region of the optical fibers 2 and 3 which is obtained after the insertion portion 101a is inserted into the body A.

The computing unit 5 includes a processor, such as a central processing unit, and a memory that stores equations or tables, and the aforementioned process in the computing unit 5 is performed by the processor.

The principle of measuring temperature with the temperature measurement device 1 will now be explained.

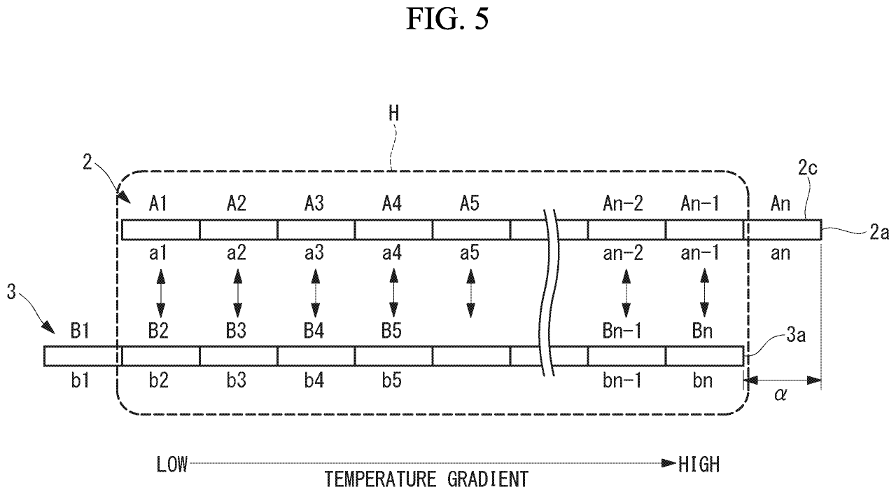

As shown in FIG. 5, a portion of the two optical fibers 2 and 3 is located in the temperature variant region H. The temperature variant region H is a region where a common temperature change occurs in the two optical fibers 2 and 3 during use of the medical device 100, and is specifically a region on the distal end side that is inserted into the body A. The length of the portion of the first optical fiber 2 located in the temperature variant region H and the length of the portion of the second optical fiber 3 located in the temperature variant region H are equal. The proximal end side of the temperature variant region H (left side in FIG. 5) is a temperature invariant region where no temperature change occurs in the optical fibers 2 and 3 during use of the medical device 100, and is specifically the proximal side region that is located outside the body.

The first optical fiber 2 in the temperature variant region H is divided equally into regions having the length a that is equal to the length of the measurement unit 2c, thereby forming n regions A1, A2, . . . , An, including the measurement unit 2c. The region An which is the region closest to the distal end is the measurement unit 2c. Similarly, the second optical fiber 3 in the temperature variant region H is divided equally into regions having the length a, thereby forming n regions B1, B2, . . . , Bn. The region Bn which is the region closest to the proximal end is a part of the temperature invariant region outside of the temperature variant region H.

The optical response values ai and bi for the regions Ai and Bi (I=1, 2, . . . , n) will now be considered. The optical response values represent changes in thermal expansion and refractive index caused by temperature change. The sums S1 and S2 of the round trip of the optical response values ai and bi in the temperature variant region H are expressed as follows. S1 is the sum of the optical response values ai of the first optical fiber 2 and S2 is the sum of the optical response values bi of the second optical fiber 3.

$$S 1 = 2 {\sum }_{i = 1}^{n} ai ,$$

$$S 2 = 2 {\sum }_{i = 1}^{n} bi$$

Here, as shown in FIG. 5, a temperature gradient occurs in the optical fibers 2 and 3 in the longitudinal direction. In addition, the region Ai and region Bi+1 are close to each other along the radial axis of the optical fibers 2 and 3. Therefore, the optical response value ai of the region Ai and the optical response value bi+1 of the region Bi+1 are equal. Accordingly, the difference ΔS between the sum S1 of the optical response value ai of the first optical fiber 2 and the sum S2 of the optical response value bi of the second optical fiber 3 is as expressed by the following equation.

$$ΔS = S 1 - S 2 = 2 {\sum }_{i = 1}^{n} ai - {\sum }_{i = 1}^{n} bi = an - b 1$$

The optical path length of the optical fibers 2 and 3 due to temperature change is changed by a change in refractive index and a change in the length of optical fibers 2 and 3 due to thermal expansion. Therefore, the sums S1 and S2 correspond to the amounts of change in optical path lengths L1 and L2 due to temperature change, respectively, and ΔS corresponds to the optical path length difference ΔL caused by temperature change. In addition, an-b1 corresponds to the amount of change in the temperature of the measurement unit 2C with respect to the temperature of the temperature invariant region. Therefore, the optical path length difference ΔL corresponds to the amount of optical path length change and the amount of temperature change in the measurement unit 2c, and the amount of temperature change in the measurement unit 2c can be estimated from the optical path length difference ΔL.

The amount of change in refractive index ΔN of the optical fibers 2 and 3 due to a temperature change of 1 °C is 1×10⁻⁵ °C. In other words, the amount of change in the optical path length of the measurement unit 2c of the length a due to a temperature change of 1 °C is α×ΔN. For example, the amount of change in the optical path length of the measurement unit 2c due to a temperature change of 1 °C is 10 μm when α=1 μm, 1 μm when α=10 cm, and 0.1 μm when α=10 mm. Since the measurement light U1 travels back and forth through the measurement unit 2c, the amount of optical path length change that occurs in the measurement unit 2c is twice (α×ΔN). When the wavelength of the measurement light U1 is 0.4 μm, even if α=10 mm, a change in temperature of 1 °C produces an optical path length change of half a wavelength of the measurement light U1.

To generate a change in optical path length, which corresponds to the wavelength of the measurement light U1, in the measurement unit 2c with a change in refractive index and thermal expansion, the following equation (1) must be satisfied. Δα is the amount of change in the length a of the measurement unit 2c due to thermal expansion.

(ΔN×α+N×Δα)×ΔT×2≈λ (1)

Since the change in the refractive index is sufficiently larger than the thermal expansion, Equation (1) can be rewritten as Equation (2) if we focus only on the change in the refractive index.

ΔN×α×ΔT×2≈λ (2)

If the amount of temperature change T is measured with an accuracy ΔT of 1/10, α=20/ΔT based on Equation (2). Here, ΔN≈1×10⁻⁵ and λ=0.4×10⁻³. Therefore, when the amount of temperature change T is 5, 20, 50, 100, or 1000, the length a of the measurement unit 2c is as follows.

| Temperature change T (°C) | Accuracy ΔT (°C) | Length α (mm) |
|---|---|---|
| 5 | 0.5 | 40 |
| 20 | 2 | 10 |
| 50 | 5 | 4 |
| 100 | 10 | 2 |
| 1000 | 100 | 0.2 |

For example, in treatment in which stones are crushed by a laser beam, when the fact that the body temperature changes by 20 °C from 37 °C is to be noted, a 20 °C temperature change can be measured with an accuracy of 2 °C by designing the length a of the measurement unit 2c to be 10 mm. In industrial applications, when measuring temperature changes of hundreds or thousands of degrees, the desired temperature change can be measured with the desired accuracy by adjusting the length a within the confines of the temperature tolerance of the optical fiber.

A laser treatment method using the laser treatment system 200 will now be explained.

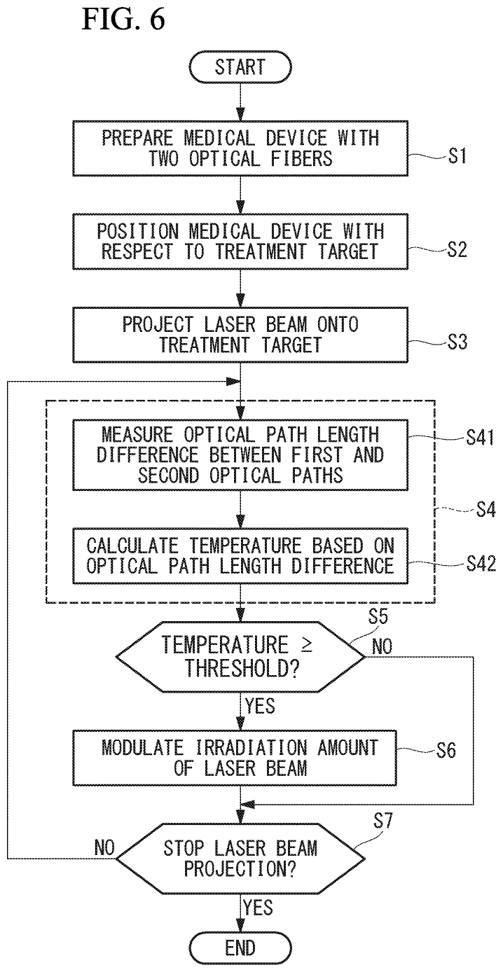

As shown in FIG. 6, the laser treatment method includes Step S1 of preparing a medical device 100 with two optical fibers 2 and 3, Step S2 of positioning the medical device 100 with respect to the treatment target B, Step S3 of projecting a therapeutic laser beam from the medical device 100 toward the treatment target B, and Step S4 of measuring the ambient temperature of the medical device 100.

In Step S1, while the temperature of the optical fibers 2 and 3 is uniform over the entire length, the two optical path lengths L1 and L2 are adjusted to the same length with the optical path length adjustment mechanism 11, and the optical path length difference ΔL is adjusted to zero. To be specific, measurement light U is emitted from the light source 7a, and the fine movement stage 11a is moved while the interference intensity of the combined light U1+U2 detected by the photodetector 10a is being observed, and the distance d is determined to be the reference distance d0 at which the interference intensity peaks. The optical path lengths L1 and L2 are adjusted either by manual operation of the fine movement stage 11a by a surgeon or other operators, or by automatic control of the fine movement stage 11a by a control device, which is not shown in the drawings, based on the interference intensity.

Next, in Step S2, the surgeon inserts the insertion portion 101a into the patient's body A and simultaneously fills the body A with perfusion fluid with the perfusion device, and positions the distal end of the insertion portion 101a in the vicinity of the treatment target B. For example, the distal end of the insertion portion 101a is positioned in the vicinity of the stone B in the ureter.

Next, in Step S3, the surgeon turns on a foot switch 105 by stepping on the foot switch 105 to emit the therapeutic laser beam V from the laser beam source 103. The laser beam V is projected from the distal end of the optical fiber 102 onto the treatment target B, so that the treatment target B is treated. For example, the projection of the laser beam V crushes the stone B. Continuously projecting the laser beam V onto the treatment target B allows the residual energy of the emitted optical energy to accumulate as thermal energy throughout the perfusion fluid present in the body A, the endoscope 101, and the optical fiber 102, resulting in a temperature increase at and around the distal end of the optical fiber 102.

While the laser beam V is being projected onto the treatment target B, Step S4, which is the temperature measurement method according to this embodiment, is performed. As shown in FIG. 6, Step S4 includes Step S41 of measuring the optical path length difference ΔL between the first optical path P1 and the second optical path P2, and Step S42 of calculating the temperature of the measurement unit 2c based on the optical path length difference ΔL.

In Step S41, the optical path length difference ΔL is measured based on the interference between the first measurement light U1 that has reciprocated along the first optical path P1 and the second measurement light U2 that has reciprocated along the second optical path P2.

To be specific, the measurement light U1 and U2 enter the first optical path P1 and the second optical path P2 from the light source unit 7 via the split-and-combine unit 8. The first measurement light U1 reciprocates along the first optical path P1 and returns to the split-and-combine unit 8. The second measurement light U2 reciprocates along the second optical path P2 and returns to the split-and-combine unit 8. The combined light U1+U2 of the first measurement light U1 and the second measurement light U2, which are combined by the split-and-combine unit 8, enters the photodetector 10a.

When the temperature of the part of the insertion portion 101a that is positioned inside the body A changes from before the insertion portion 101a was inserted into the body A, both the optical path length L1 of the first optical path P1 and the optical path length L2 of the second optical path P2 change. At this time, the amount of change in optical path length L1 and the amount of change in optical path length L2 differ from each other due to the fact that the measurement unit 2c, which is the distal end of the first optical fiber 2, extends beyond the distal end of the second optical fiber, resulting in an optical path length difference ΔL.

To detect the optical path length difference ΔL, the second optical path length L2 is changed using the optical path length adjustment mechanism 11, and the amount of change Δd in the optical path length L2 at which interference between the first measurement light U1 and second measurement light U2 occurs is detected. To be specific, the distance d between the split-and-combine unit 8 and the coupler 9b is changed between the distance d− and the distance d+ by moving the fine movement stage 11a, to detect the distance d0′ at which the interference intensity of the combined light U1+U2 detected by the photodetector 10a peaks, thereby detecting the amount of change Δd in distance d0′ from the reference distance d0. The distance −d is smaller than the distance d0 and the distance d+ is larger than the distance d0.

Next, in Step S42, the amount of temperature change T in the measurement unit 2c corresponding to the amount of change Δd is calculated using the computing unit 5, and the current temperature of the measurement unit 2c is calculated based on the amount of temperature change T and the temperature of the measurement unit 2c observed before insertion. The calculated amount of temperature change T and the current temperature are displayed on the display 6.

Continuously projecting the laser beam V onto the treatment target B increases the temperature at and around the distal end of the optical fiber 102. To prevent excessive temperature increase, the laser treatment method may further include Step S5 of comparing the temperature of the measurement unit 2c with a predetermined threshold, and if the temperature is equal to or more than the predetermined threshold (YES in Step S5), Step S6 of modulating the irradiation amount of the laser beam V projected onto the treatment target B. The modulation of the irradiation amount includes, for example, increasing, reducing, or modulating at least one of the peak power, pulse width, and repetition frequency of a pulsed laser beam V.

The surgeon observes the current temperature of the measurement unit 2c displayed on the display 6, and when the temperature exceeds a predetermined threshold, he or she reduces the amount of irradiation by modulating the amount of irradiation. For example, the surgeon stops the projection of the laser beam V by turning off the foot switch 105. Instead of stopping the projection of the laser beam V, the surgeon may change the position of the optical fiber 102. For example, in Step S6, the surgeon may move the optical fiber 102 radially by moving the insertion portion 101a radially. This allows the target spot of projection of the laser beam V to be changed to prevent excessive temperature increase due to continued projection of the laser beam V onto the same spot. Alternatively, the surgeon may move the optical fiber 102 away from the treatment target B by retracting the insertion portion 101a or the optical fiber 102 toward the proximal end. This can weaken the intensity of the laser beam V projected onto the treatment target B.

The laser treatment method may further include the step of re-modulating the amount of irradiation of the laser beam V if, after Step S6, the measured temperature drops below a predetermined threshold (YES in Step S5). For example, the surgeon increases the amount of irradiation by modulating the amount of irradiation. The modulation of the amount of irradiation also includes, for example, modulating at least one of the peak power, pulse width, and repetition frequency of a pulsed laser beam V. The modulation of the amount of irradiation may be performed by the surgeon manually operating the light source 103 or by a control device, not shown in the drawing, automatically controlling the light source 103.

Thus, the temperature measurement device 1 of this embodiment measures the temperature at and around the distal end of the insertion portion 101a, using two optical fibers 2 and 3 positioned outside the insertion portion 101a. The accuracy of temperature measurement is determined by the length a of the measurement unit 2c, which is barely affected by deformation of the optical fibers 2 and 3. Accordingly, appropriately designing the length a of the measurement unit 2c allows the desired high measurement accuracy to be easily achieved, thereby accurately measuring the temperature change and temperature at and around the distal end of the insertion portion 101a.

In addition, since generally-used optical fibers can be used as the optical fibers 2 and 3, higher measurement accuracy can be achieved with a less expensive configuration than when optical fibers with FBGs are used.

The thin optical fibers 2 and 3 can be added to the medical device 100 with little or no increase in the outer diameter of the medical device 100. Thus, the temperature measurement device 1 can be suitably mounted on the medical device 100 with a small diameter.

### Second Embodiment

A temperature measurement device and medical device system according to the second embodiment of the present invention will now be described. In this embodiment, the same components as in the first embodiment will be denoted by the same reference numerals as the corresponding ones, and explanations thereof will be omitted.

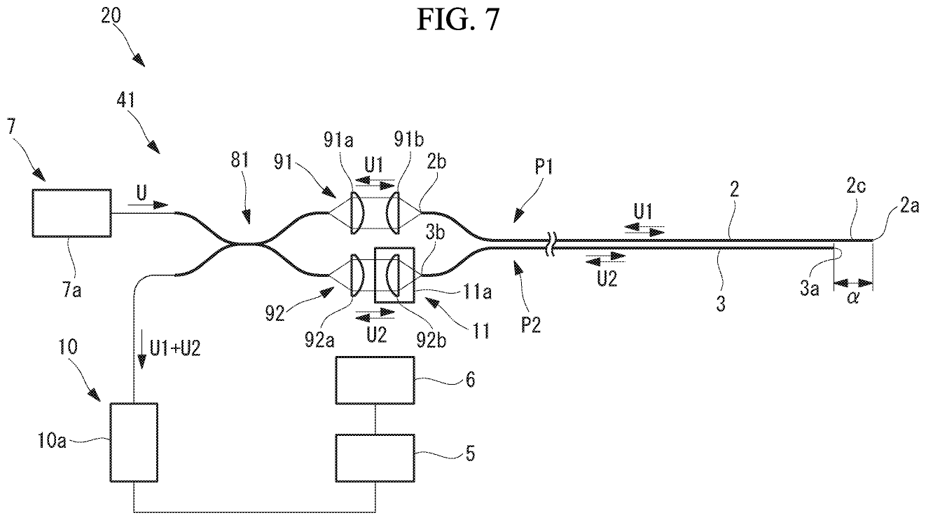

The medical device system of this embodiment includes a medical device 100 and a temperature measurement device 20. As shown in FIG. 7, the temperature measurement device 20 differs from the temperature measurement device 1 in that it has an optical path length difference measuring unit 41 instead of an optical path length difference measuring unit 4.

The temperature measurement device 20 includes a first optical fiber 2, a second optical fiber 3, an optical path length difference measuring unit 41 for measuring the optical path length difference ΔL, a computing unit 5, and a display 6.

The optical path length difference measuring unit 41 includes a light source unit 7, a split-and-combine unit 81, two couplers 91 and 92, a detection unit 10, and an optical path length adjustment mechanism 11.

The split-and-combine unit 81 is an optical fiber coupler and is positioned between the light source unit 7 and the proximal ends 2b and 3b of the two optical fibers 2 and 3. The split-and-combine unit 81 splits the measurement light U incident from the light source unit 7 into measurement light U1 traveling along the first optical path P1 and second measurement light U2 traveling along the second optical path P2. The split-and-combine unit 8 also combines the first measurement light U1 that has reciprocated along the first optical path P1 and the second measurement light U2 that has reciprocated along the second optical path P2.

To be specific, the optical fiber coupler 81 has two input ports and two output ports. One input port is connected to the light source unit 7, and the other input port is connected to the detection unit 10. One output port is connected to the proximal end 2b via the first coupler 91, and the other output port is connected to the proximal end 3b via the second coupler 92.

The coupler 91 optically couples one output port to the proximal end 2b, and the coupler 92 optically couples the other output port to the proximal end 3b. To be specific, the coupler 91 has a first lens 91a located on the split-and-combine unit 81 side and a second lens 91b located on the optical fiber 2 side. The coupler 92 has a first lens 92a located on the split-and-combine unit 81 side and a second lens 92b located on the optical fiber 3 side. The first lenses 91a and 92a convert the measurement light U1 and U2 emitted from the split-and-combine unit 81 into collimated light, respectively, and the second lenses 91b and 92b focus the collimated light onto the proximal ends 2b and 3b, respectively.

The first optical path P1 is the optical path between the split-and-combine unit 81 and the distal end 2a of the first optical fiber 2. In other words, the first measurement light U1 returns to the split-and-combine unit 81 from the split-and-combine unit 81 via the coupler 91, the proximal end 2b, the distal end 2a, the proximal end 2b, and the coupler 91 in this order.

The second optical path P2 is the optical path between the split-and-combine unit 81 and the distal end 3a of the second optical fiber 3. In other words, the second measurement light U2 returns to the split-and-combine unit 81 from the split-and-combine unit 81 via the coupler 92, the proximal end 3b, the distal end 3a, the proximal end 3b, and the coupler 92, in this order.

The second lens 92b of the coupler 92 and the proximal end of the second optical fiber 3 are fixed to the fine movement stage 11a of the optical path length adjustment mechanism 11, and the fine movement stage 11a, the second lens 92b, and the proximal end of the second optical fiber 3 move together. Accordingly, the distance d between the split-and-combine unit 81 and the second lens 92b can be slightly changed by the fine movement of the fine movement stage 11a, thereby fine-tuning the optical path length L2.

Other configurations, actions, and effects of the temperature measurement device 20 are the same as those of the temperature measurement device 1, and the description thereof will therefore be omitted.

### Third Embodiment

The temperature measurement device and medical device system according to the third embodiment of the present invention will now be described. In this embodiment, the same components as in the first embodiment will be denoted by the same reference numerals as the corresponding ones, and explanations thereof will be omitted.

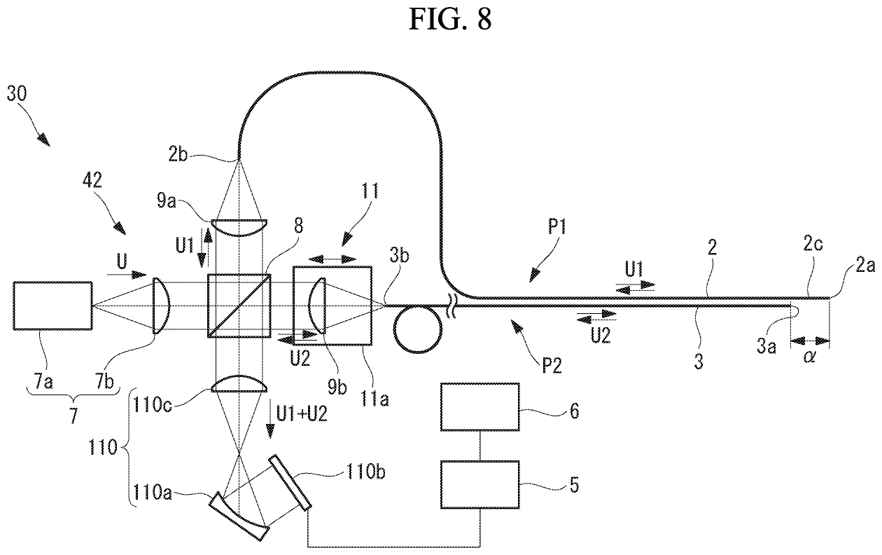

The medical device system according to this embodiment includes a medical device 100 and a temperature measurement device 30. As shown in FIG. 8, the temperature measurement device 30 differs from the temperature measurement device 1 in that it has a detection unit 110 instead of a detection unit 10.

The temperature measurement device 30 includes a first optical fiber 2, a second optical fiber 3, an optical path length difference measuring unit 42, a computing unit 5, and a display 6.

The optical path length difference measuring unit 42 includes a light source unit 7, a split-and-combine unit 8, two couplers 9a and 9b, a detection unit 110, and an optical path length adjustment mechanism 11.

The detection unit 110 includes a spectrometer that spectrally splits combined light U1+U2 and detects the intensity of combined light U1+U2 at each wavelength. To be specific, the spectrometer consists of a diffraction grating 110a that spectrally splits combined light U1+U2, and a linear sensor 110b that is positioned in the direction of the wavelength of the combined light U1+U2 that has been spectrally split and detects the light intensity at each wavelength.

The detection unit 110 includes a lens 110c positioned between the split-and-combine unit 8 and the diffraction grating 110a, and the lens 110c may focus the combined light U1+U2 between the split-and-combine unit 8 and the diffraction grating 110a.

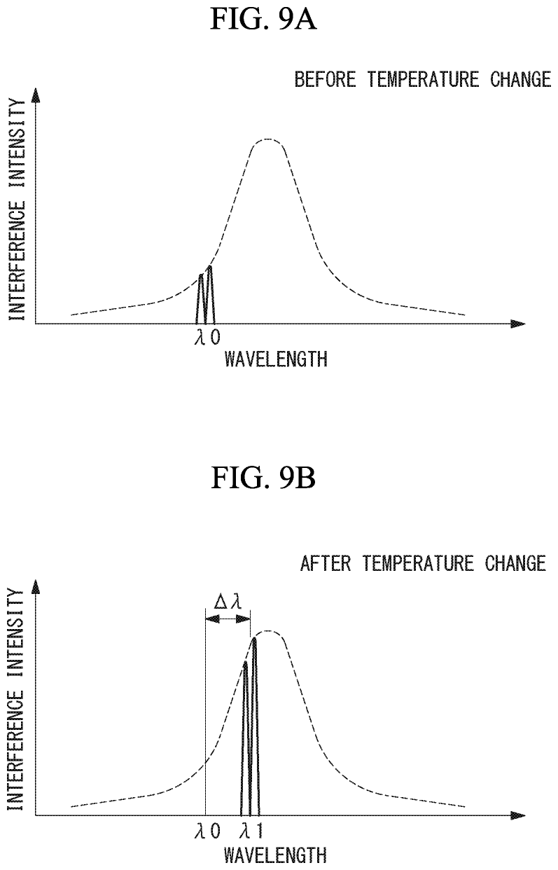

The interference between the first measurement light U1 and the second measurement light U2 forms an interference pattern on the linear sensor 110b, resulting in an interference intensity distribution at a specific wavelength, as shown in FIGS. 9A and 9B. FIG. 9A shows the interference intensity distribution observed when the optical path lengths L1 and L2 are equal, and FIG. 9B shows the interference intensity distribution observed when the optical path length difference ΔL is caused by temperature change. In FIGS. 9A and 9B, the horizontal axis represents wavelength and the vertical axis represents optical intensity. The measurement light U1 and U2 are light having a wavelength width, and the wavelength at which the interference intensity distribution is generated changes according to the optical path length difference ΔL. Thus, the optical path length difference ΔL can be estimated from the amount of change in wavelength Δλ at which the interference intensity distribution occurs.

A laser treatment method using a laser treatment system 200 will now be described.

The laser treatment method of this embodiment differs from the laser treatment method described in the first embodiment in Steps S1 and S41.

In Step S1, the two optical path lengths L1 and L2 are adjusted so that they become equal while the temperature of the optical fibers 2 and 3 is uniform over the entire length. To be specific, the fine movement stage 11a is moved while the interference intensity distribution detected by the linear sensor 110b is observed, and the distance d is determined to be the reference distance d0 at which the interference intensity distribution is detected at a predetermined reference wavelength Δ0.

In Step S41, the optical path length difference ΔL is measured based on the interference between the first measurement light U1 that has reciprocated along the first optical path P1 and the second measurement light U2 that has reciprocated along the second optical path P2.

To be specific, the measurement light U1 and U2 enter the first optical path P1 and the second optical path P2 from the light source unit 7 via the split-and-combine unit 8. The first measurement light U1 reciprocates along the first optical path P1 and returns to the split-and-combine unit 8. The second measurement light U2 reciprocates along the second optical path P2 and returns to the split-and-combine unit 8.

The combined light U1+U2 of the first measurement light U1 and the second measurement light U2, which are combined by the split-and-combine unit 8, is spectrally split by the diffraction grating 110a. Next, based on the light intensity at each wavelength detected by the linear sensor 110b, the wavelength λ1 at which the interference intensity distribution is generated is detected, and the amount of change in wavelength λ1 from the reference wavelength Δ0, Δλ, is detected.

In the next Step S42, the amount of temperature change T in the measurement unit 2c corresponding to the amount of change Δλ is calculated in the computing unit 5.

Other configurations, actions, and effects of the temperature measurement device 30 are the same as those of the temperature measurement device 1, and the description thereof will therefore be omitted.

### Fourth Embodiment

The temperature measurement device and medical device system according to the fourth embodiment of the present invention will now be described. In this embodiment, the same components as in the first embodiment will be denoted by the same reference numerals as the corresponding ones, and explanations thereof will be omitted.

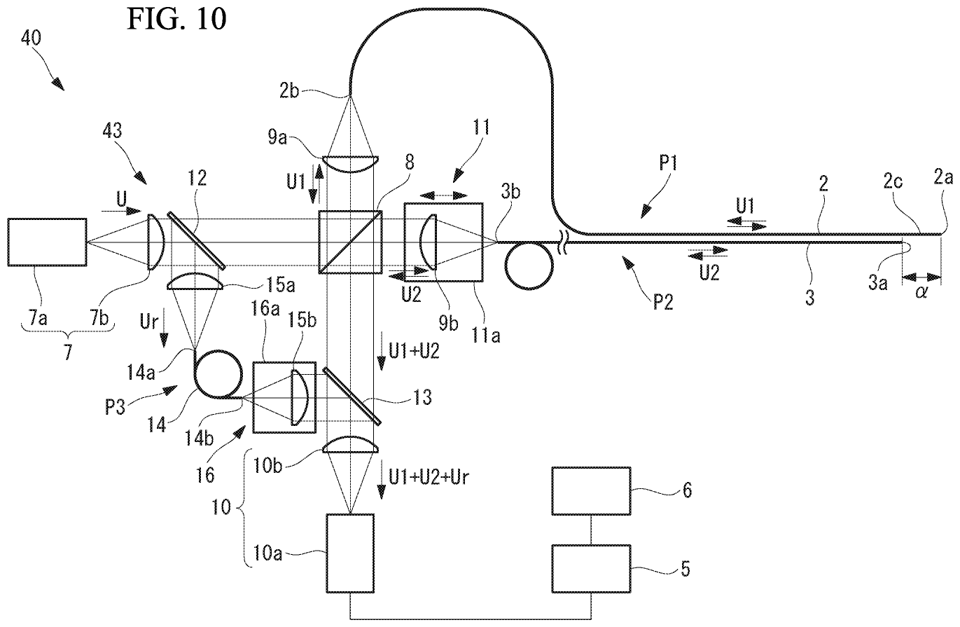

The medical device system according to this embodiment includes a medical device 100 and a temperature measurement device 40. As shown in FIG. 10, the temperature measurement device 40 differs from the temperature measurement device 1 in that it has an optical path length difference measuring unit 43 instead of an optical path length difference measuring unit 4.

The temperature measurement device 40 includes a first optical fiber 2, a second optical fiber 3, an optical path length difference measuring unit 43 for measuring the optical path length difference ΔL, a computing unit 5, and a display 6.

The optical path length difference measuring unit 43 includes a light source unit 7, a split-and-combine unit 8, two couplers 9a and 9b, a detection unit 10, an optical path length adjustment mechanism 11, a reference light separating unit 12, a reference light combining unit 13, a third optical fiber 14, two couplers 15a and 15b, and another optical path length adjustment mechanism 16.

The reference light separating unit 12 is positioned between the light source unit 7 and the split-and-combine unit 8 and separates part of the measurement light U as reference light Ur. The reference light separating unit 12 is, for example, a beam sampler.

The reference light combining unit 13 is positioned between the split-and-combine unit 8 and the detection unit 10 and combines the reference light Ur with the combined light U1+U2. The reference light combining unit 13 is, for example, a combining plate such as a translucent mirror that reflects the reference light Ur and transmits the measurement light U1 and U2.

The third optical fiber 14 is positioned between the reference light separating unit 12 and the reference light combining unit 13.

The coupler 15a is positioned between the reference light separating unit 12 and the incident end 14a of the third optical fiber 14, and optically couples the reference light separating unit 12 and the incident end 14a. The coupler 15b is positioned between the reference light combining unit 13 and the emitting end 14b of the third optical fiber 14, and optically couples the reference light combining unit 13 and the emitting end 14b. To be specific, the couplers 15a and 15b are convex lenses. The coupler 15a focuses the reference light Ur, which is collimated light incident from the reference light separating unit 12, onto the incident end 14a, and the coupler 15b converts the reference light Ur, which is diffused light incident from the emitting end 14b, into collimated light which is then incident into the reference light combining unit 13.

The optical path length adjustment mechanism 16 fine-tunes the third optical path length L3 of the third optical path P3 of the reference light Ur from the reference light separating unit 12 to the reference light combining unit 13. The optical path length L3 is designed to be twice each of the optical path lengths L1 and L2. In practice, however, it is not easy to make the optical path length L3 twice each of the optical path lengths L1 and L2 due to errors in the length of the optical fiber 14.

The optical path length adjustment mechanism 16 includes a fine movement stage 16a that is capable of fine movement with wavelength-order resolution in the direction along the optical axis between the emitting end 14b and the coupler 15b. The coupler 15b is fixed to the fine movement stage 16a, and the fine movement stage 16a and the coupler 15b move together. Accordingly, the optical path length L3 can be fine-tuned by fine movement of the fine movement stage 16a.

In this embodiment, the combined light U1+U2+Ur of the first measurement light U1, the second measurement light U2, and the reference light Ur enters the photodetector 10a.

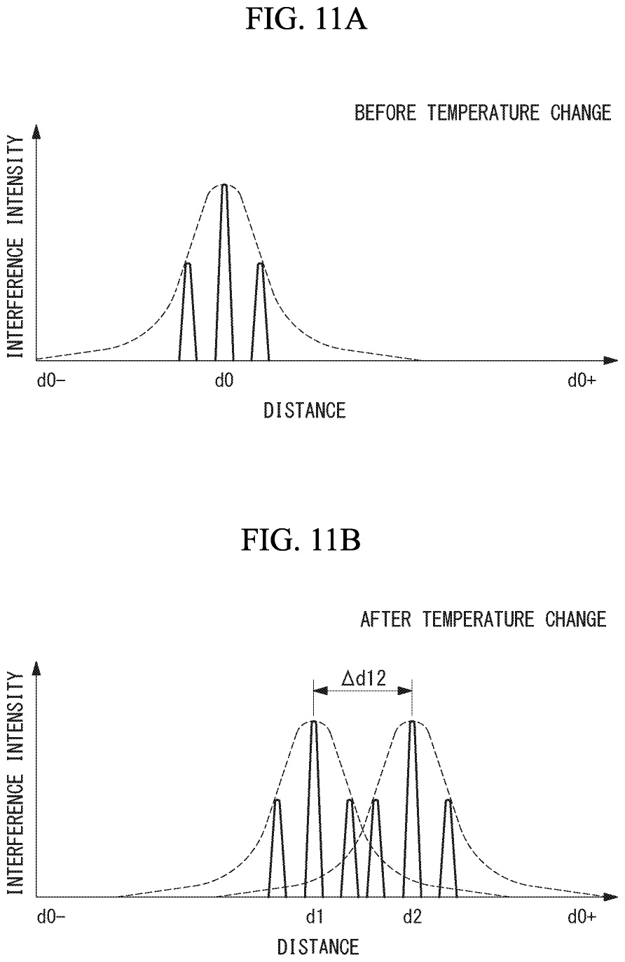

When the three optical path lengths L1, L2, and L3 are equal, as shown in FIG. 11A, a single interference occurs when the three beams of light U1, U2, and U3 interfere mutually and intensify each other.

On the other hand, when the optical path length difference ΔL is occurring according to the amount of temperature change T in the measurement unit 2c, two interference intensity distributions occur as shown in FIG. 11b. One interference intensity distribution is due to the interference between the first measurement light U1 and the reference light Ur, and the other interference intensity distribution is due to the interference between the second measurement light U2 and the reference light Ur. The distance difference Δd12 between the two interference intensity distributions varies according to the optical path length difference ΔL. Therefore, the temperature of the measurement unit 2c can be estimated from the distance difference Δd12 between the distance d1 of the fine movement stage 16a at which one interference intensity distribution occurs and the distance d2 of the fine movement stage 16a at which the other interference intensity distribution occurs. The distances d1 and d2 are the distances between the emitting end 14b and the coupler 15b.

The computing unit 5 stores an equation or table showing the relationship between the distance difference Δd12 and the amount of temperature change T, and obtains the distance difference Δd12 and the corresponding amount of temperature change T from the equation or table.

The laser treatment method using a laser treatment system 200 will now be described.

The laser treatment method of this embodiment differs from the laser treatment method that has been described in the first embodiment in Steps S1 and S41.

In Step S1, while the temperature of the optical fibers 2, 3, and 14 is uniform over the entire length, the two optical path lengths L1 and L2 are adjusted to equal using the optical path length adjustment mechanism 11, and the optical path length L3 is adjusted to twice each of the optical path lengths L1 and L2 using the optical path length adjustment mechanism 16.

In Step S41, the optical path length difference ΔL is measured based on the interference between the first measurement light U1 and the reference light Ur and the interference between the second measurement light U2 and the reference light Ur.

To be specific, the measurement light U1 and U2 enter the first optical path P1 and the second optical path P2 from the light source unit 7 via the split-and-combine unit 8. The first measurement light U1 reciprocates along the first optical path P1 and returns to the split-and-combine unit 8. The second measurement light U2 reciprocates along the second optical path P2 and returns to the split-and-combine unit 8. Also, reference light Ur enters the third optical path P3 from the light source unit 7 via the reference light separating unit 12. The reference light Ur travels along the third optical path P3 and is combined with the combined light U1+U2 in the reference light combining unit 13, and the combined light U1+U2+Ur enters the photodetector 10a.

When the third optical path length L3 of the reference light Ur is changed by the optical path length adjustment mechanism 16 to detect the optical path length difference ΔL, the optical path length difference between the first optical path length L1 and the third optical path length L3 is obtained from the interference intensity generated by the first measurement light U1 and the reference light Ur, as shown in FIG. 11B. At the same time, the optical path length difference between the second optical path length L2 and the third optical path length L3 is obtained from the interference intensity generated by the second measurement light U2 and the reference light Ur, and the difference Δd12 between these optical path length differences is detected. To be specific, the optical path length L3 of the reference light Ur is changed by moving the fine movement stage 16a, between a distance d− and a distance d+ with respect to the distance of the reciprocation of each of the first optical path length L1 and the second optical path length L2, thereby detecting the two distances d1 and d2 at which the interference intensity of the combined light U1+U2+Ur detected by the photodetector 10a peaks, and calculating the difference Δd12 between the distances d1 and d2.

In the next Step S42, the amount of temperature change T in the measurement unit 2c corresponding to the distance difference Δd12 is calculated in the computing unit 5.

In the first and second embodiments, in Step S1, it is necessary to detect and store the reference distance d0 obtained when the optical path length difference ΔL is zero. In contrast, this embodiment has an advantage over the first and second embodiments in that it does not need to store the reference distance d0.

Other configurations, actions, and effects of the temperature measurement device 40 are the same as those of the temperature measurement device 1, and the description thereof will therefore be omitted.

In the first to fourth embodiments described above, the two optical fibers 2 and 3 are positioned on the outer surface of the insertion portion 101a in parallel with the longitudinal direction of the insertion portion 101a; however, the positioning of the two optical fibers 2 and 3 is not limited to this and can be modified as needed.

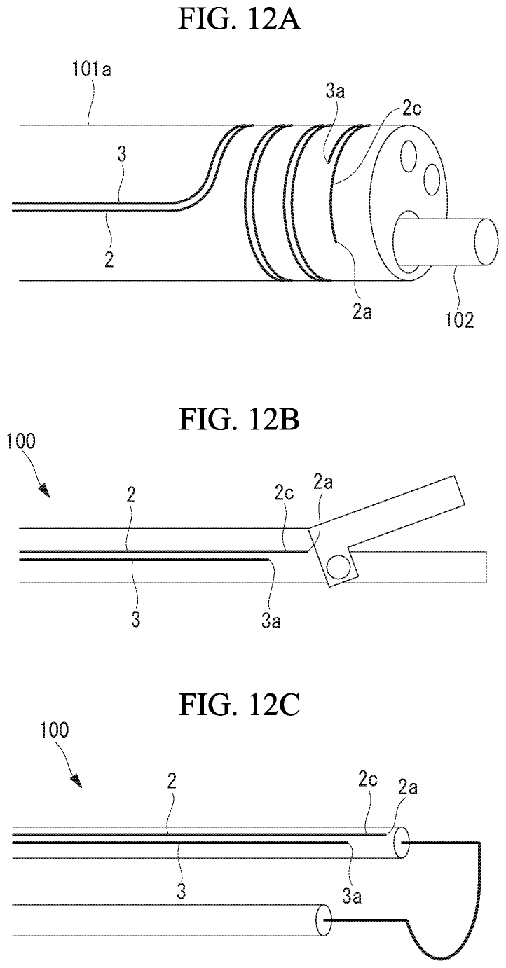

For example, as shown in FIG. 12A, the two optical fibers 2 and 3 may be spirally wound around the side surface of the distal end of the insertion portion 101a. The larger the length a of the measurement unit 2c, the more accurate the temperature measurement. Winding the optical fibers 2 and 3 in a circumferential direction allows the measurement unit 2c with a large length a to be easily positioned in the vicinity of the distal end of the insertion portion 101a, thereby easily increasing the accuracy of temperature measurement.

The optical fibers 2 and 3 need only to be positioned in the medical device 100 so that at least the measurement unit 2c is exposed from the medical device 100. For example, the portion closer to the proximal end than the measurement unit 2c may be positioned inside the insertion portion 101a.

In the first to fourth embodiments described above, the medical device 100 is supposed to be a laser treatment device; however, the temperature measurement device 1 can be applied to other medical devices 100. FIGS. 12B and 12C show examples of applying the temperature measurement device to other medical devices 100. The medical device 100 in FIG. 12B is forceps, pincer, or other treatment instrument. The medical device 100 in FIG. 12C is an electrical treatment instrument with electrodes.

### REFERENCE SIGNS LIST

1, 20, 30, 40 Temperature measurement device
2 First optical fiber
2c Measurement unit
3 Second optical fiber
4, 41, 42, 43 Optical path length difference measuring unit
5 Computing unit
7 Light source unit
8 Split-and-combine unit
10, 110 Detection unit
110a, 110b Spectrometer (diffraction grating, linear sensor)
11, 16 Optical path length adjustment mechanism
12 Reference light separating unit
13 Reference light combining unit
100 Medical device
200 Laser treatment system
U, U1, U2 Measurement light
Ur Reference light
V Laser beam
P1 First optical path
P2 Second optical path

1. A method for measuring temperature at a distal end of a medical device, using a first optical fiber having a measurement unit at a distal end thereof and a second optical fiber, the method comprising:
in response to application of heat proximate the measurement unit, measuring an optical path length difference between a first optical path including the first optical fiber and a second optical path including the second optical fiber based at least in part on a first measurement light transmitted at least in part by the first optical fiber and a second measurement light transmitted at least in part by the second optical fiber;
calculating a temperature associated with the measurement unit based on the optical path length difference; and
displaying the calculated temperature.

2. The method according to claim 1, wherein in the measuring,
measuring the optical path length difference based on interference between the first measurement light that has reciprocated along the first optical path and the second measurement light that has reciprocated along the second optical path.

3. The method according to claim 2, wherein in the measuring,
changing an optical path length of one of the first optical path and the second optical path; and
detecting an amount of change in the optical path length of the one of the first optical path and the second optical path at which an interference intensity distribution between the first measurement light and the second measurement light occurs.

4. The method according to claim 2, wherein in the measuring,
spectrally splitting a combined light of the first measurement light and the second measurement light; and
detecting an amount of change in wavelength at which an interference intensity distribution between the first measurement light and the second measurement light occurs.

5. The method according to claim 1, wherein in the measuring,
measuring the optical path length difference based on interference between the first measurement light that has reciprocated along the first optical path and reference light, and interference between the second measurement light that has reciprocated along the second optical path and the reference light.

6. The method according to claim 1, further comprising fine-tuning an optical path length of the first optical path or an optical path length of the second optical path with wavelength-order precision.

7. The method according to claim 1, further comprising setting a length of the measurement unit based on a temperature change amount desired to be measured.

8. The method of claim 1, further comprising setting an optical path length of the first optical path to be equal to an optical path length of the second optical path prior to the application of heat.

9. The method of claim 8, further comprising setting: a first wavelength of the first measurement light to be equal to a second wavelength of the second measurement light and (ii) a first phase of the first measurement light to be equal to a second phase of the second measurement light.

10. The method of claim 1, wherein the temperature associated with the measurement unit is calculated based on (i) a temperature change determined based on the optical path length difference resulting from the application of heat and (ii) an initial temperature associated with the measurement unit prior to the application of heat.

11. The method of claim 10, further comprising determining the temperature change based on an experimentally determined relationship between the temperature change and a change in the optical path length difference.

12. A device comprising:
a first optical fiber and a second optical fiber positioned in a medical device, the first optical fiber having a measurement unit at a distal end thereof;
an optical path length difference measuring unit configured to measure an optical path length difference between a first optical path including the first optical fiber and a second optical path including the second optical fiber based at least in part on a first measurement light transmitted at least in part by the first optical fiber and a second measurement light transmitted at least in part by the second optical fiber; and
a processor comprising hardware, the processor being configured to:
calculate a temperature associated with the measurement unit based on the optical path length difference; and
control a display to display the calculated temperature.

13. The device according to claim 12,
wherein the optical path length difference measuring unit comprises:
a light source configured to emit measurement light;
a splitter positioned between the light source and the first optical path and the second optical path, the splitter being configured to split the measurement light into the first measurement light traveling along the first optical path and the second measurement light traveling along the second optical path, and to combine the first measurement light that has reciprocated along the first optical path and the second measurement light that has reciprocated along the second optical path; and
a detector configured to detect an intensity of combined light of the first measurement light and the second measurement light that have been combined by the splitter.

14. The device according to claim 13, further comprising an optical path length adjustment mechanism configured to adjust an optical path length of one of the first optical path and the second optical path.

15. The device according to claim 13, wherein the detector comprises a spectrometer configured to spectrally split the combined light and to detect an intensity at each wavelength of the combined light.

16. The device according to claim 13, wherein:
the optical path length difference measuring unit comprises:
a beam sampler configured to separate part of the measurement light as reference light; and
a combining plate, positioned between the splitter and the detector, and configured to combine the reference light with the combined light;
the detector is configured to detect an intensity of a combined light of the first measurement light, the second measurement light, and the reference light that have been combined by the combining plate; and
an optical path length of a third optical path along which the reference light travels from the beam sampler to the combining plate is equal to the optical path length of the first optical path and the optical path length of the second optical path.

17. The device according to claim 12, further comprising an optical path length adjustment mechanism configured to fine-tune an optical path length of the first optical path or an optical path length of the second optical path with wavelength-order precision.

18. The device according to claim 12, a length of the measurement unit is set based on a temperature change amount desired to be measured.
